(19) United States
(12) Patent Application Publication
(10) Pub. No.: US 2012/0294823 A1
(43) Pub. Date: Nov. 22, 2012

(54) Method for preparing silk sericin-PVA scaffold using genipin as crosslinking agent

(76) Inventor: Pornanong Aramwit, Patumwan (TH)

(21) Appl. No.: 13/068,581

(22) Filed: May 16, 2011

(51) Int. Cl. A61K 31/765; A61P 17/02; C12N 5/02

(52) U.S. Cl. 424/78.06; 435/396

(57) ABSTRACT

A method for preparing a porous-three-dimensional scaffold good for tissue engineering is described. Sericin forms a three-dimensional scaffold with PVA after freeze-drying having glycerin as a plasticizer and genipin as natural crosslinking agent to help making a strong and stable matrix. Adding glycerin into scaffold gives good uniformity and porosity. Smaller pore sizes and better uniformity are obtained as the concentration of genipin in the scaffold increases. Glycerin retains a high moisture content to allow the presence of water molecule in the matrix structure. Adding genipin results in a higher degree of crosslinking within the scaffold. Crosslinking using genipin is most beneficial in preparing scaffold possesses the best biological and physical properties for wound healing. The present invention describes method for preparing crosslinked matrix whose composition can be appropriately tuned to obtain matrix with desirable characteristics for biological applications.

### FIELD OF THE INVENTION

The present invention relates generally to method for preparing silk sericin-PVA scaffold using genipin as crosslinking agent having plasticizer to form product with good properties. The invention includes scaffold composed of silk sericin and polyvinyl alcohol having plasticizer(s) and a natural crosslinking agent.

### BACKGROUND OF THE INVENTION

Accidental damage to the epidermis by ulcers, burns or other traumatic incidents may result in a series of morbid consequences that restrict epidermal regeneration. In the case of wounds that extend entirely through the dermis, skin substitutes such as xenografts, allografts and autografts need to be employed for wound healing. The design of substrates to allow specific biological interactions is demanding, particularly in the case of tissue engineered skin substitutes. Natural biomaterials such as collagen, silk and chitosan have received increasing attention in the field of biomedical engineering due to their unique properties, including non-toxicity, biodegradability and biocompatibility. Porous-three-dimensional scaffolds that can provide a framework for cells to attach, proliferate and form their extracellular matrix play an important role in manipulating cell functions in this approach. Since a suitable scaffold should possess the specific structure of the tissue it replaces and must be capable in turn of being replaced in time via the ingress of new cells, the choice of material is of prime concern. However, natural biomaterials themselves are normally unable to meet all these requirements. Polymer blending is a useful technique for modifying the properties of a single polymer. Silk sericin, a natural hydrophilic polymer extracted from silk cocoons during the degumming process, is non-toxic to fibroblast cells and enhances wound healing by promoting collagen production in wounds. Sericin is mainly comprised of serine and aspartic acid with strong polar side chains, thus enabling easy copolymerization and capable of being blended with other polymers to produce biocompatible materials with desirable properties. Sericin itself forms fragile materials that are not suitable for use in medical applications, but it has been demonstrated (Mandal et al., Acta Biomater. 5 (2009) 3007-3020) that after blending with gelatin, silk sericin can form a scaffold and be a good candidate for tissue engineering applications. Polyvinyl alcohol (PVA) (a synthetic polymer with good biocompatibility, low toxicity and good mechanical properties) was blended with sericin. A crosslinking process is also believed to improve the permeability as well as the mechanical properties of proteins. Genipin (Methyl (1R,2R,6S)-2-hydroxy-9-(hydroxymethyl)-3-oxabicyclo[4.3.0]nona-4,8-diene-5-carboxylate) is found in traditional Chinese medicine and is extracted from gardenia fruit. It is an effective naturally occurring crosslinking agent that can react with amino acids or proteins containing residues with primary amine groups such as lysine, hydroxylysine or arginine. Sung et al. (J. Biomater. Sci. Polym. Ed. 10 (1999) 751-771 and J. Biomed. Mater. Res. 46 (1999) 520-530) investigated the cytotoxicity, feasibility and biocompatibility of genipin for tissue fixation and found that genipin is 10,000 times less cytotoxic than the commonly used glutaraldehyde. In addition, the treatment of animal wounds by genipin-crosslinked glue induced significantly lower inflammatory responses and more rapid recovery than those treated by aldehyde-crosslinked glues. Glycerin, a commonly used plasticizer, has been mixed to improve silk film properties and also helps to reduce phase separation between silk and PVA in the blend. Glycerin content in blend films is important for the control of silk secondary structural transitions and influencing the mechanical properties of the films. After mixing with silk, glycerin molecules interact with silk chains via intermolecular forces, mostly hydrogen bonds between hydroxyl groups of glycerin and amide groups of silk.

Kato, Tsujimoto, and Yamada (U.S. Pat. No. 7,763,448) disclosed porous body obtained only by gelling an aqueous solution of a material consisting of sericin followed by freezing and thawing with no use of any crosslinking agent. Thus, it is very difficult if not impossible to control pore-size or the degree of crosslink to allow desirable strength of the porous body and makes it very easy to collapse. Such product requires much improvement to use it in practice.

The present invention discloses method for preparing silk sericin and PVA scaffolds, with genipin as crosslinking and glycerin as plasticizer, is of great advantage in tissue engineering due to their low toxicity and the degree of crosslink can be designed to give best product for wound healing of desirable strength.

### SUMMARY OF THE INVENTION

A method for preparing a porous-three-dimensional scaffold is described. The scaffold shows several advantages for tissue engineering since it provides a good framework for cells to attach, proliferate and form an extracellular matrix. Sericin forms a three-dimensional scaffold with PVA after freeze-drying but with a fragile, structure. Glycerin (as a plasticizer) and genipin (a crosslinking agent) help making a strong and stable matrix. Adding glycerin into scaffold gives good uniformity and porosity. Smaller pore sizes and better uniformity were obtained as the concentration of genipin in the scaffold increased. Glycerin retains a high moisture content to allow the presence of water molecule in the matrix structure. Adding genipin results in a higher degree of crosslinking within the scaffold, while further adding of glycerin significantly increases degree of crosslinking and water retention. Genipin enhances the moisture absorption capacity of the scaffold and extended the time taken to reach equilibrium of sericin release from scaffold. After immersing the sericin/PVA scaffold into water, the scaffold completely dissolved within an hour, whereas the scaffolds containing glycerin or glycerin with 0.1% genipin swelled 8 and 11 times, respectively after 6 h. Crosslinking using genipin is most beneficial in preparing scaffold possesses the best biological and physical properties for wound healing. The present invention describes method for preparing scaffold which can be appropriately tuned to obtain scaffolds with desirable characteristics for biological applications.

### BRIEF DESCRIPTION OF THE DRAWINGS

FIG. 1 shows percentage of crosslinks in sericin/PVA/glycerin scaffold with various concentrations of genipin.

FIG. 2 shows percentage weight change of sericin/PVA scaffold with and without glycerin and different concentrations of genipin after placing into high humidity (˜80%) environment.

FIG. 3 shows swelling of sericin/PVA scaffold with and without glycerin and various concentrations of genipin after immersion in water.

FIG. 4 shows the amount of protein released from the scaffolds.

### DETAILED DESCRIPTION OF THE PREFERRED EMBODIMENTS

The present invention described method for preparing silk sericin-PVA scaffold using genipin as crosslinking agent. Silk sericin is extracted from pieces (about 5 mm²) of cocoons from silkworms (Bombyx mori) using a high temperature and pressure degumming technique. Pieces of silkworm cocoons are mixed with purified water (1 g of dry silk cocoon: 30 mL of water) and autoclaved at 120° C. for 60 min. After filtration through a membrane to remove fibroin, sericin solution was concentrated until the desired concentration (approximately 7% (w/v)) is achieved. PVA (molecular weight 77,000-82,000) is dissolved at 80° C. with constant stirring for about 4 h until it is completely dissolved to a concentration of 6% (w/v). Genipin is dissolved in ethyl alcohol to give a solution at a concentration of 20% (w/v). Sericin solution and PVA solution with or without glycerin are blended together at room temperature for at least 30 min to make a final wet composition of 3% (w/v) sericin, 2% (w/v) PVA and 1% (w/v) glycerin. Genipin solution is added to the mixed solution of sericin, PVA and glycerin to make final concentrations of 0.01-0.1% w/v and stirred for 5 min, which is then poured into a petri-dish and frozen at −20° C., and followed by lyophilization for 72 h.

Mixing sericin and PVA aqueous solution with or without glycerin results in homogeneous mixture. Genipin does not cause gel formation or significant increase in viscosity of sericin/PVA and glycerin solution (the viscosity of sericin/PVA/glycerin and sericin/PVA/glycerin with genipin solution were <0.3 dPa s). Scaffold composed of various concentrations of sericin or PVA, both ranging from 1 to 5% w/v, are observed for their physical properties. Up to 10% w/v may also be tested. The most suitable concentration of sericin and PVA to give homogenous and stable matrix is sericin, PVA and glycerin at a ratio of concentration 3, 2 and 1% w/v, respectively on wet weight basis. It can easily form a scaffold after freeze-drying and appears as a smooth and homogenous material. After freeze-drying, final weight of the scaffold do not show significant difference compared with theoretical weight. Various scaffolds composed of sericin (3% (w/v))/PVA (2% (w/v))/glycerin (1% (w/v)) and genipin at different concentrations are obtained. Without genipin, both sericin/PVA and sericin/PVA/glycerin scaffolds appear off-white in color, which is the natural color of the silk cocoon. Genipin changes the color of the scaffold to pale blue (at a low concentration, 0.01%) and dark blue (at a high concentration, 0.1%) due to natural color of genipin. The sericin/PVA scaffold is rigid and less flexible compared to the scaffold with glycerin and genipin.

Table 1 shows the pore size distribution of sericin scaffolds. The sericin/PVA scaffold has a high pore size variation compared with the other types of scaffold while the sericin/PVA/glycerin scaffold exhibited smaller pore sizes and better uniformity compared with the sericin/PVA scaffold. Adding genipin into the scaffolds results in an increase in the mean pore size. However, the size of the porous diameter decreases and uniformity increases with increasing genipin concentration. All scaffolds are highly porous, which is quite suitable in terms of their use as tissue engineering material.

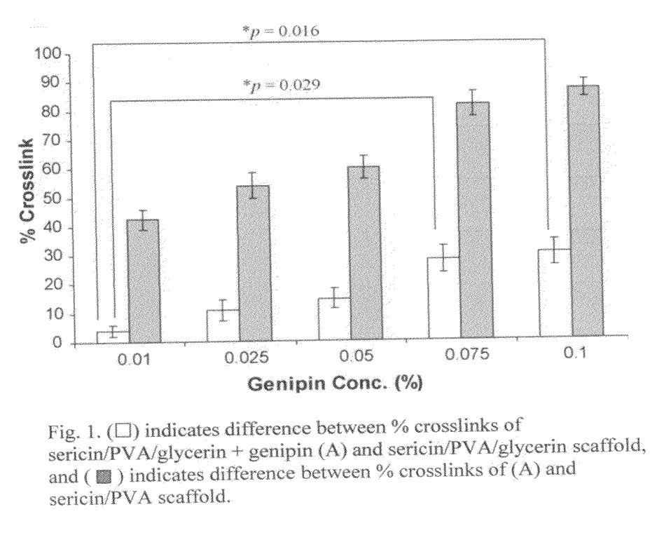

Primary amino groups in peptides and proteins is determined using TNBS (2,4,6-trinitrobenzene sulfonic acid) as a UV chromophore. FIG. 1 shows the percentage of crosslinks in the sericin/PVA/glycerin scaffolds with various concentrations of genipin from 0.01 to 0.1% compared with that of the sericin/PVA and sericin/PVA/glycerin scaffolds (Aramwit et al. Int. J. Biol. Macromol. 47 (2010) 668-675). Higher concentrations of genipin in the scaffold results in a higher degree of crosslinking and fewer free ε-amino groups. Addition of 0.1% genipin to the scaffold increases the degree of crosslinking by approximately 30% compared with the sericin/PVA/glycerin scaffold, and up to 80% when compared with the sericin/PVA scaffold. Genipin at 0.01% concentration showed significant difference in degree of crosslinking when compared with the scaffold composed of 0.075 and 0.1% genipin. The crosslinking mechanism of genipin and sericin containing amine is not well understood. It is suggested that the reaction occurs with amino acid lysine, hydroxylysine and arginine of sericin which possess the primary amine side chain (Park et al. J. Agric. Food Chem. 50 (2002) 6511-6514.).

The reaction occurs through a nucleophilic attack of the primary amine on the C3 carbon of genipin. This causes an opening of the dihydropyran ring. An attack on the resulting aldehyde group by the secondary amine then follows. The final step in the formation of crosslinking is believed to be the dimerization produced by radical reactions. This indicates that genipin can form both intramolecular and intermolecular crosslinks. Glycerin can enhance the crosslinking in the sericin/PVA scaffold, which indicates that plasticizers such as glycerin can significantly enhance the formation of crosslinks within caseinates (milk proteins chains) (Brault et al. J. Agric. Food Chem. 45 (1997) 2964-2969.). Similar behaviors were observed with other plasticizer such as propylene glycol and triethylene glycol. The present invention shows that genipin can effectively crosslink sericin.

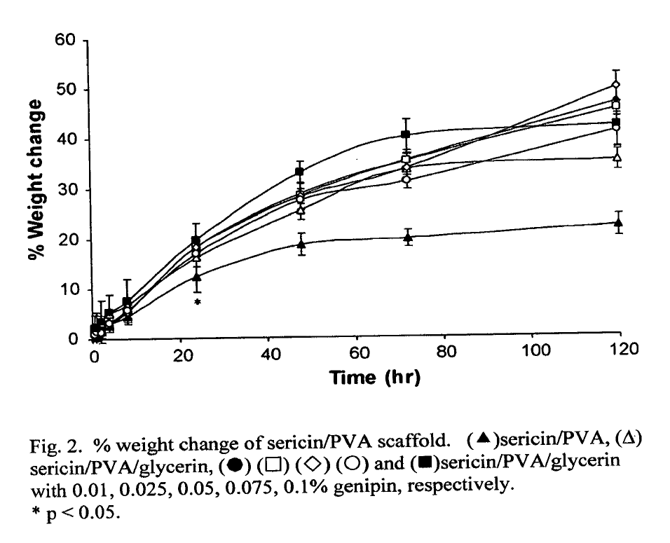

The percentage weight change of the scaffolds after placing them in a high humidity environment is shown in FIG. 2. The sericin/PVA scaffold has the lowest ability to absorb moisture, but adding glycerin significantly increases this ability. This may partly be due to the moisture absorption capacity of glycerin itself. After 24 h, sericin/PVA scaffold absorbed moisture significantly less compared with scaffolds composed of genipin (p=0.003, 0.002, 0.002, 0.022 and 0.000 for the case of 0.01, 0.025, 0.05, 0.075 and 0.1% genipin, respectively). Genipin also enhances the moisture absorption capacity of the sericin/PVA scaffold and extends the time taken to reach equilibrium. The time required to attain equilibrium swelling is longer for the sericin/PVA/glycerin scaffold with genipin at a concentration between 0.01 and 0.075% compared with the sericin/PVA scaffold with and without glycerin. Without genipin, the moisture absorption capacity of the sericin/PVA and sericin/PVA/glycerin scaffold reached the equilibrium within 3 days while those containing genipin had not reached equilibrium even after 5 days. Genipin concentration of the scaffolds between 0.01 and 0.1% produced an approximately 10% difference in weight change from moisture absorption.

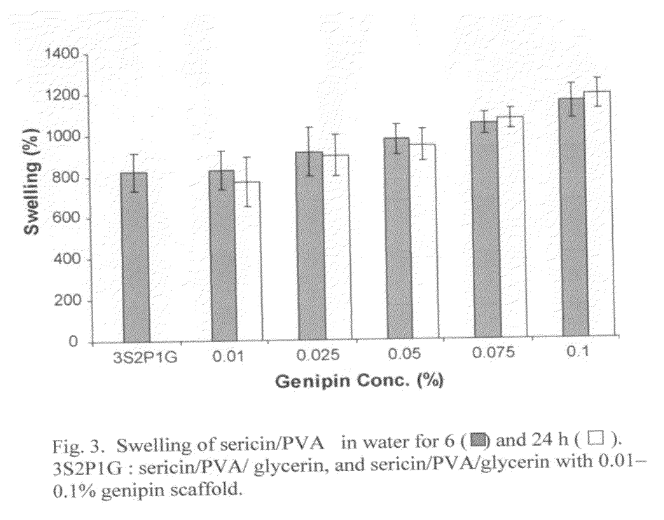

The swelling of the sericin/PVA scaffold with and without glycerin and various concentrations of genipin after immersion in water for 6 and 24 h is shown in FIG. 3. The percentage swelling of the scaffolds at equilibrium was calculated using the following equation:

$$\% swelling = \frac{Wt - W 0}{W 0} \times 100$$

where W0 is the weight of the dried test sample and Wt is the weight of the swollen test sample.

The sericin/PVA scaffold was completely dissolved within 1 h. There was an 8-fold swelling of the sericin/PVA/glycerin scaffold compared with the initial weight after 6 h immersion and this scaffold was completely dissolved within 24 h. The swelling of sericin/PVA/glycerin with genipin increased over a period of time and was directly related to the percentage weight of genipin added to the scaffold base. At 0.1% genipin, the swelling after 6 and 24 h immersion was about 11 and 12 times that of the initial stage, respectively. A higher degree of genipin oligomerization resulted in a porous network with higher swelling properties. The longer equilibrated moisture absorption time (FIG. 2) resulted in the higher swelling ratio (FIG. 3). This may be due to the flexible structure of the scaffold containing genipin, which was characterized by slow water sorption but a high water holding capacity. The swelling properties at 6 and 24 h were not significantly different, because the three-dimensional scaffold allows its total surface area to interact with the water molecules during the initial swelling. Thus, adding glycerin alone to the sericin/PVA scaffold is not enough to make scaffolds that are stable in an aqueous solution for 24 h. Genipin or other crosslinking agents are necessary in order to provide solid material suitable for biological applications.

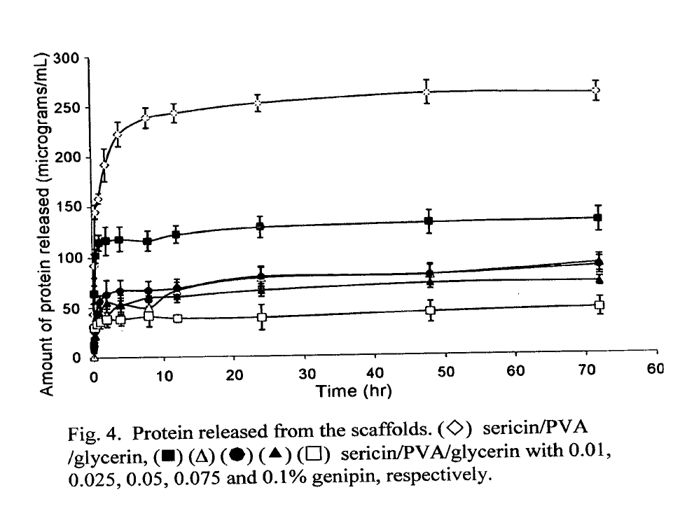

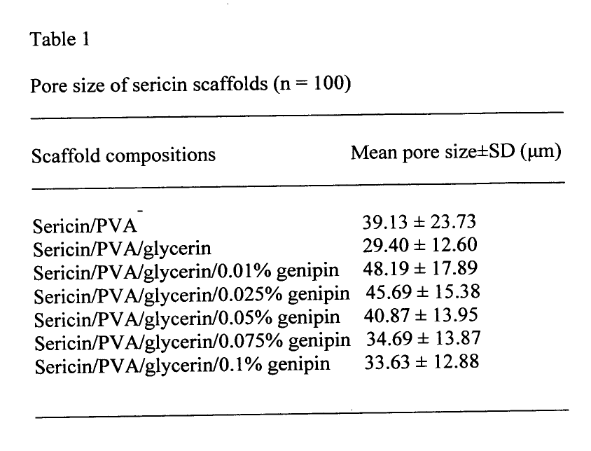

Amount of protein released from the scaffolds is showed in FIG. 4. The sericin/PVA scaffold completely dissolved and released all sericin in less than 30 min (data not shown). Sericin/PVA/glycerin scaffold without genipin released the highest amount of sericin, while higher genipin concentration led to the release of a lower amount of protein. Maximum protein leaching from all scaffolds was observed within 48 h. The fraction of protein released from the sericin/PVA/glycerin scaffold was approximately 4%, with values of about 1.03 and 0.04% in the case of scaffolds with 0.01 and 0.1% genipin, respectively. As sericin can activate collagen production in wounds, low levels of sericin released from the scaffold will be beneficial for healing and, at the same time, the matrix would also be stable. The sericin/PVA scaffold released large amounts of sericin, where the structure was completely degraded after immersion for a few hours. Since free sericin molecules that remain non-crosslinked contribute to the leached-out protein fraction, the sericin/PVA/glycerin scaffold that had the lowest degree of crosslinking compared to the scaffold with genipin exhibited higher sericin release, resulting in structural collapse, which makes it not useful for further application. Adding genipin to the scaffold leads to lower sericin release and a more intact structure which would be beneficial in terms of wound healing and tissue engineering. The fraction of protein released from the scaffold was quite low, with a maximum of about 4% in the scaffold without the crosslinking agent, while scaffolds with genipin released an even smaller amount of protein. Lower amount of PVA, approximately 33-40% (mean 36.7±2.6%, n=3), is released from sericin/PVA/glycerin with 0.10% genipin scaffold under the same condition. The significant lower amount of PVA released from scaffold containing high concentration of genipin (higher degree of crosslink) may be due to the higher entrapment of PVA between sericin chain, resulting in less available amount of this polymer to be released (p<0.01). Taking into account, the high swelling and the amount of protein as well as PVA released, erosion might be the degradation behavior of sericin/PVA/glycerin scaffolds. Since small amount of sericin and some portions of PVA were released from scaffold, part of the scaffold structure still maintained and stable even after 48 h immersion.

The method of preparing silk sericin-PVA scaffold using genipin as crosslinking agent disclosed is of great benefit to tissue engineering and a great inventive step as to the silk sericin-PVA scaffold itself with glycerin and with genipin as crosslinking agent can release small amount of sericin to activate collagen production in wounds. Yet, more could be done where biomolecules or other small functioning molecules of therapeutic use can be crosslinked or conjugated to the scaffold through primary amine groups to expand its usefulness.

It will be understood that modifications can be made in the above description without departing from the scope of this invention by one of ordinary skill in the art. It is accordingly intended that all matter contained in the above description be interpreted as descriptive and illustrative rather than in a limiting sense.

It is also to be understood that the following claims are intended to cover all of the generic and specific features of the invention as described herein, and all statements of the scope of the invention which, as a matter of language, might be said to fall therebetween.

We claim:

1. A method for preparing silk sericin-PVA scaffold using genipin as crosslinking agent comprises
step of extracting silk sericin using a high temperature and pressure degumming technique, where pieces of silkworm cocoons are mixed with purified water and autoclaved at 120° C. for 60 min., filtering through a membrane to remove fibroin, concentrating of sericin solution;
step of dissolving PVA (molecular weight 77,000-82,000) at 80° C. with constant stirring for about 4 h to obtain a concentration of 6% (w/v);
step of dissolving genipin in ethyl alcohol to give a solution at a concentration of 20% (w/v);
step of blending sericin solution and PVA solution with glycerin together at room temperature for at least 30 min to make a final mixture having wet composition of 3% (w/v) sericin, 2% (w/v) PVA and 1% (w/v) glycerin;
step of adding genipin solution to the mixed solution of sericin, PVA and glycerin to make final concentrations of 0.01-0.1% w/v of genipin and stirred for 5 min, and poured into a petri-dish, frozen at −20° C., and lyophilizing for 72 h where various scaffolds composed of sericin (3% (w/v))/PVA (2% (w/v))/glycerin (1% (w/v)) and genipin at different concentrations are obtained for use in tissue engineering.

2. A method for preparing a crosslinked matrix comprising at least one natural polymer, one synthetic polymer, one plasticizer and one natural crosslinking agent comprising:
step of preparing solution of said natural polymer to give a concentration of 1-10% w/v;
step of dissolving said synthetic polymer to give solution at concentration of 1-10% w/v;
step of dissolving said natural crosslinking agent in appropriate solvent to give a concentration up to 20% w/v;
step of blending solution of said natural polymer and said synthetic polymer with plasticizer, preferably glycerin;
step of adding solution of said natural crosslinking agent to the mixed solution, stirring, and pouring into a container, frozen at −20° C., and lyophilizing for 72 h to obtain crosslinked matrix having natural crosslinking agent at different concentrations.

3. A method for preparing a crosslinked matrix of claim 2 where said crosslinked matrix is used in tissue engineering especially wound healing and where leaching of small amount of protein or peptide from said matrix helps activating collagen production in wounds and where bioactive molecules may be crosslinked or conjugated to said matrix for medical use.
